(19) United States
(12) Patent Application Publication
(10) Pub. No.: US 2021/0061226 A1
(43) Pub. Date: Mar. 4, 2021

(54) VEHICLE AND CONTROL METHOD THEREOF

(71) Applicants: Hyundai Motor Company, Seoul (KR); Kia Motors Corporation, Seoul (KR)

(72) Inventor: Yongseok Jeong, Yongin (KR)

(21) Appl. No.: 16/742,534

(22) Filed: Jan. 14, 2020

(30) Foreign Application Priority Data
Aug. 27, 2019 (KR) 10-2019-0105323

(51) Int. Cl. B60R 25/24; B60R 25/102; B60R 25/20; A61B 5/08; A61B 5/00; B60R 25/31; G06Q 50/26

(52) U.S. Cl. CPC B60R 25/246; B60R 25/102; B60R 25/241; B60R 25/209; G06Q 50/265; A61B 5/4845; B60R 25/31; A61B 5/6893; A61B 5/0022; A61B 5/082

(57) ABSTRACT

A vehicle is provided and includes a digital key system that receives a result of alcohol drinking authentication from a user terminal in which at least one application is installed and an engine management system that generates a control signal for activating or deactivating engine driving of the vehicle based on the result. A controller receives a result for an alcohol drinking status of a first driver based on the alcohol drinking authentication performed by the user terminal and initiates a call to a second driver when the first driver is determined to be in the alcohol drinking status. The controller receives a result for alcohol drinking status of the second driver and operates the engine management system according to the alcohol drinking status of the second driver.

### CROSS-REFERENCE TO RELATED APPLICATION(S)

This application is based on and claims priority under 35 U.S.C. § 119 to Korean Patent Application No. 10-2019-0105323, filed on Aug. 27, 2019, the disclosure of which is incorporated by reference in its entirety.

### TECHNICAL FIELD

The present invention relates to a vehicle and a control method of the vehicle, and more particularly, to a vehicle and a control method of the vehicle, which prevent drunk driving.

### BACKGROUND

Various countries have regulations or are forming regulations regarding breathalyzers installed within vehicles. Accordingly, vehicle companies are developing a vehicle breathalyzer. In recent years, a vehicle to which a user terminal, that is, digital key is applied has been produced. The user terminal, that is, digital key executes an authentication program (application) in the user terminal capable of wireless communication with a vehicle to complete user authentication and controls various functions of the vehicle.

Various applications may be installed in the digital key, and a function of breathalyzers may be provided according to the content of the application. In view of the variety of applications, a vehicle that performs wireless communication with digital keys may provide new ways to effectively prevent drunk driving.

### SUMMARY

Therefore, the present disclosure provides a vehicle and a control method of vehicle capable of preventing drunk driving and forcing a request for surrogate driving in an alcohol drinking status.

In accordance with an aspect of the disclosure, a vehicle may include: a digital key system configured to receive a result of alcohol drinking authentication from a user terminal in which at least one application is installed; an engine management system configured to generate a control signal for activating or deactivating engine driving of the vehicle based on the result; and a controller including at least one processor; wherein the controller is configured to receive a result for an alcohol drinking status of a first driver based on the alcohol drinking authentication performed by the user terminal, initiate a call to a second driver when the first driver is determined to be in an alcohol drinking status, receive a result for alcohol drinking status of the second driver and operate the engine management system according to the alcohol drinking status of the second driver.

The at least one application may include a digital key application, a breathalyzer application and a surrogate driving application. The controller may be configured to initiate the call to the second driver when the digital key application is linked with the breathalyzer application and the surrogate driving application. Alternately, the at least one application may include a digital key application and the controller may be configured to suggest an installation of the breathalyzer application to the first driver when the digital key application is not linked with a breathalyzer application. According to another exemplary embodiment, the at least one application may include a digital key application and a breathalyzer application and the controller may be configured to output the surrogate driving site result through the search portal when the digital key application is not linked with the surrogate driving application.

The controller may be configured to receive a result of the alcohol drinking status of the second driver and deactivate the engine management system when the second driver is determined to be in an alcohol drinking status. The controller may also be configured to receive a result of the alcohol drinking status of the second driver and activate the engine management system when the second driver is determined to not be in an alcohol drinking status. The at least one application may include at least one of a digital key application, a breathalyzer application, a surrogate driving application and a search portal application. The controller may be configured to maintain deactivation of the engine management system when the first driver is determined to be in an alcohol drinking status. The controller may be configured to operate the engine management system to change from deactivation to activation in response to detecting that the second driver reaches the vehicle.

In accordance with an aspect of the disclosure, a control method of a vehicle may include: receiving a result of alcohol drinking authentication from a user terminal in which at least one application is installed; generating a control signal for activating or deactivating engine driving of the vehicle based on the result; receiving a result for an alcohol drinking status of a first driver based on the alcohol drinking authentication performed by the user terminal; initiating a call to a second driver when the first driver is determined to be in an alcohol drinking status; receiving a result for alcohol drinking status of the second driver; and operating the engine management system according to the alcohol drinking status of the second driver.

The at least one application may include a digital key application, a breathalyzer application and a surrogate driving application and the initiating of the call to a second driver may include: initiating the call to the second driver when the digital key application is linked with the breathalyzer application and the surrogate driving application. Alternately, the at least one application may include a digital key application and the initiating of a call to a second driver may include: suggesting an installation of a breathalyzer application to the first driver when the digital key application is not linked with the breathalyzer application.

According to another exemplary embodiment, the at least one application may include a digital key application and a breathalyzer application and the initialization of a call to a second driver may include: outputting the surrogate driving site result through the search portal when the digital key application is not linked with the surrogate driving application. The operating of the engine management system may include: receiving a result of the alcohol drinking status of the second driver and deactivating the engine management system when the second driver is determined to be in an alcohol drinking status.

The operating the engine management system may further include: receiving a result of the alcohol drinking status of the second driver and activating the engine management system when the second driver is determined to not be in an alcohol drinking status. The at least one application may include at least one of a digital key application, a breathalyzer application, a surrogate driving application and a search portal application. The operating the engine management system may include: maintaining deactivation of the engine management system when the first driver is determined to be in an alcohol drinking status and changing from deactivation to activation in response to detecting that the second driver reaches the vehicle.

In accordance with an aspect of the disclosure, a non-transitory computer readable recording medium comprising program causing the computer to execute steps of: receiving a result of alcohol drinking authentication from a user terminal in which at least one application is installed; generating a control signal for activating or deactivating engine driving of the vehicle based on the result; receiving a result for an alcohol drinking status of a first driver based on the alcohol drinking authentication performed by the user terminal; initiating a call to a second driver when the first driver is determined to be in an alcohol drinking status; receiving a result for alcohol drinking status of the second driver; and operating the engine management system according to the alcohol drinking status of the second driver. The at least one application may include a digital key application, a breathalyzer application, a surrogate driving application and a search portal application.

### BRIEF DESCRIPTION OF THE DRAWINGS

These and/or other aspects of the disclosure will become apparent and more readily appreciated from the following description of the exemplary embodiments, taken in conjunction with the accompanying drawings of which:

FIG. 1 illustrates an exterior configuration of a vehicle according to one exemplary embodiment of the disclosed invention;

FIG. 2 illustrates an internal configuration of a vehicle according to an exemplary embodiment of the disclosed invention;

FIG. 3 is a control block diagram of a vehicle according to one exemplary embodiment of the disclosed invention;

FIG. 4 is a view for explaining an application installed in the user terminal according to an exemplary embodiment of the present invention; and

FIGS. 5 and 6 are flowcharts of a control method of a vehicle according to an exemplary embodiment of the disclosed invention.

### DETAILED DESCRIPTION

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor. The memory is configured to store the modules and the processor is specifically configured to execute said modules to perform one or more processes which are described further below.

Furthermore, control logic of the present disclosure may be embodied as non-transitory computer readable media on a computer readable medium containing executable program instructions executed by a processor, controller/control unit or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Like reference numerals refer to like elements throughout the specification. Not all elements of embodiments of the disclosure will be described, and description of what are commonly known in the art or what overlap each other in the embodiments will be omitted. The terms as used throughout the specification, such as “˜ part,” “˜ module,” “˜ member,” “˜ block,” etc., may be implemented in software and/or hardware, and a plurality of “˜ parts,” “˜ modules,” “˜ members,” or “˜ blocks” may be implemented in a single element, or a single “˜ part,” “˜ module,” “˜ member,” or “˜ block” may include a plurality of elements.

It will be understood that when an element is referred to as being “connected” to another element, it can be directly or indirectly connected to the other element, wherein the indirect connection includes “connection” via a wireless communication network. Further, when it is stated that a layer is “on” another layer or substrate, the layer may be directly on another layer or substrate or a third layer may be disposed therebetween.

It will be understood that, although the terms first, second, third, etc., may be used herein to describe various elements, it should not be limited by these terms. These terms are only used to distinguish one element from another element. As used herein, the singular forms “a,” “an” and “the” are intended to include the plural forms as well, unless the context clearly indicates otherwise.

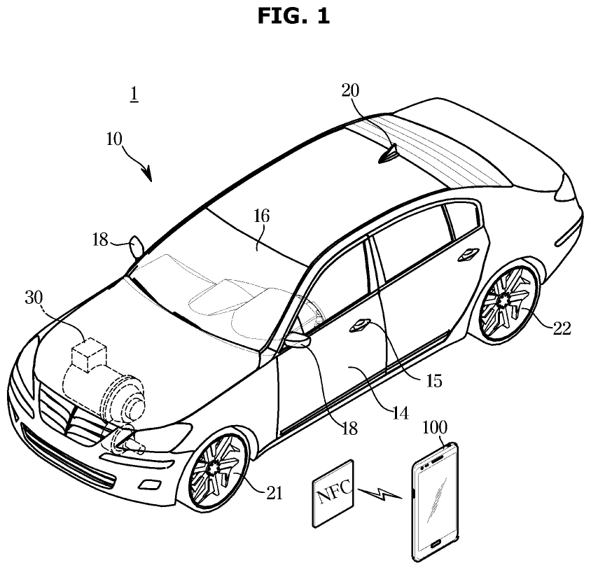

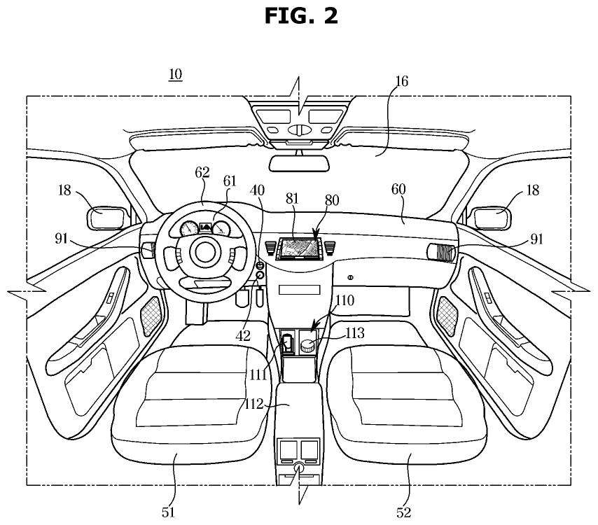

Hereinafter, the operation principles and exemplary embodiments of the disclosure will be described with reference to the accompanying drawings. FIG. 1 illustrates an exterior configuration of a vehicle according to one exemplary embodiment of the disclosed invention. FIG. 2 illustrates an internal configuration of a vehicle according to an exemplary embodiment of the disclosed invention.

As shown in FIG. 1, a vehicle 1 according to an exemplary embodiment of the disclosed invention may include a vehicle body 10 forming an outer shape of the vehicle, a door 14 to shield the inside of the vehicle body 10 from the outside, a windshield 16 which provides the driver with a view in front of the vehicle 1, a side mirror 18 which provides the driver with a view behind the vehicle 1, wheels 21 and 22 for moving the vehicle 1, and a drive device 30 for rotating wheels 21 and 22

The door 14 is rotatably provided on the left and right sides of the vehicle body 10 to allow the driver to enter the vehicle 1. The door 14 may shield the inside of the vehicle 1 from the outside when closed. The door 14 may be locked and unlocked using the door handle 15. The locking and unlocking method of the door handle 15 includes a method in which a driver approaches the vehicle 1 and directly manipulates a button or a lever of the door handle 15. In addition, the locking and unlocking method of the door handle 15 includes a method of locking and unlocking remotely using a remote controller or the like at a position away from the vehicle 1.

The door handle 15 may include a near field communication (NFC) communication module (not shown) configured to perform NFC communication with the user terminal 100. The windshield 16 may be disposed on the front upper side of the vehicle body 10 to allow the driver to acquire visual information in front of the vehicle 1 and may be implemented as a windshield glass. In addition, the side mirrors 18 are mounted on the left and right sides of the vehicle body 10, and allow the driver inside the vehicle 1 to acquire visual information on the side and rear of the vehicle 1.

In addition, the vehicle 1 may include an antenna 20 mounted on an upper surface of the vehicle body 10. The antenna 20 receives broadcast and communication signals such as telematics, DMB, digital TV, and global positioning system (GPS). The antenna 20 may be a multifunctional antenna for receiving various types of broadcast/communication signals or a single function antenna for receiving any one broadcast/communication signal. Telematics is a combination of Telecommunications and Informatics, and is defined as a next-generation information service for vehicles through a combination of the IT industry and the automotive industry. Telematics technology may use mobile communication technology and location tracking technology (GPS) to collect vehicle information and may provide various multimedia services such as air conditioning setting and remote start, detection of an accident or theft of the vehicle 1, guidance of a driving route, and inquiry of the state of the vehicle 1.

The wheels 21 and 22 include a front wheel 21 provided at the front of the vehicle body 10 and a rear wheel 22 provided at the rear of the vehicle body 10. The drive device 30 applies a rotational force to the front wheel 21 or the rear wheel 22 to move the vehicle body 10 forward or rearward. The drive device 30 may employ an engine configured to generate rotational force by burning fossil fuel or a motor configured to generate rotational force by receiving power from a battery (not shown). The vehicle 1 to which the disclosed invention is applied may be an electric vehicle such as an electric vehicle (EV), a hybrid electric vehicle (HEV), or a fuel cell electric vehicle (FCEV).

As shown in FIG. 2, the vehicle 1 may include seats 51 and 52 for occupants to sit on, a steering wheel 62 provided at the driver's seat 51, a cluster 61 toward the front of the vehicle body 10 and configured to display operation information of the vehicle 1, a dashboard 60 connected to the cluster 61 to install various devices for manipulating the vehicle 10, therein.

Specifically, the dash board 60 may protrude from the lower portion of the windshield 16 toward the seats 51 and 52, and the driver may operate various devices installed on the dash board 60 while driving. On the other hand, the dashboard 60 at the driver's seat 51 side may be formed with a key groove 40 for inserting a remote control device (not shown; e.g., a fob key). In the key groove 40 a remote control device capable of starting the vehicle 1 may be inserted.

In addition, the dash board 60 in which the key groove 40 is formed may be provided with a start button 42 for turning the vehicle 1 on and off. When the remote control device is inserted into the key groove 40 or when the authentication between the remote control device and the vehicle 1 is successful via the wireless communication network, the start of the vehicle 1 may be turned on by pressing the start button 42 of the user. For example, various devices provided in the dash board 60 may include an audio-video-navigation (AVN) device 80, in the center fascia, which is the central area of the dashboard 60, vents 91 of an air conditioning device (not shown) provided on the side of the touch screen 81 of the AVN device 80 and various input devices provided at the bottom of the AVN device 80.

The AVN device 80 may be configured to perform audio, video and navigation functions according to a passenger's operation. The AVN device 80 may be connected to a controller configured to operate the electric vehicle 1 as a whole, that is, a head unit. The AVN device 80 may be configured to perform two or more functions. For example, the AVN device 80 may be configured to turn on audio to play music recorded on a compact disc (CD) or a universal serial bus (USB) and simultaneously perform a navigation function. In addition, the AVN device 80 may be configured to turn on the video to display a digital media broadcasting (DMB) image and simultaneously perform a navigation function.

The AVN device 80 may be configured to display a screen related to an audio function, a screen related to a video function, or a screen related to a navigation function through the touch screen 81. The touch screen 81 according to an example may be configured to display the charging state of the vehicle 1. The touch screen 81 may be implemented as a liquid crystal display (LCD) panel, a light emitting diode (LED) panel, an organic light emitting diode (OLED) panel, or the like. The touch screen 81 may be configured to perform a screen display function and an input function of an instruction or a command.

The touch screen 81 may be configured to output a screen having a predetermined image to the outside or receive an instruction or a command according to an operating system (OS) for driving and operating the AVN device 80 and an application running in the AVN device 80. In addition, the touch screen 81 may be configured to output information that may be output from the user terminal 100 by applying a technique such as mirroring or casting. The touch screen 81 may also be configured to display a basic screen according to the running application. The basic screen refers to a screen displayed by the touch screen 81 when no touch manipulation is performed. The touch screen 81 may be configured to display a touch manipulation screen based on the situation. The touch manipulation screen refers to a screen capable of receiving a touch manipulation from a user.

An input method of the touch screen 81 may use a resistive touch screen type to detect user's touch operation, a capacitive touch screen method to detect the touch operation of the user using the capacitive coupling effect, an optical touch screen method using infrared rays or an ultrasonic touch screen method using ultrasonic waves. In addition, various input methods may be used, and the present invention is not limited thereto. The touch screen 81 is a device that enables the user to interact with the AVN device 80 provided in the vehicle 1. Further, the touch screen 81 is a device that receives a user command by using a touch interaction or the like, and receives a user command by selecting a letter or a menu displayed on the touch screen 81.

Particularly, the AVN device 80 may be referred to as a navigation terminal or a display device, and may be referred to in various terms used by those skilled in the art by being integrated with a telematics unit (not shown). In addition, the AVN device 80 may be connected to a communication terminal such as a smart phone, a portable multimedia player (PMP), a MPEG audio layer-3 (MP3) player, a personal digital assistant (PDA) by Equipping with a USB port, and may be configured to play audio and video files.

Furthermore, vent apertures 91 of an air conditioning device (not shown) may be provided at both sides of the touch screen 81 in the dash board 60. The air conditioning device refers to a device for automatically adjusting an air conditioning environment including an indoor/outdoor environmental condition of the vehicle 1, air intake/exhaust, circulation, cooling/heating state, or for adjustment in response to a user's control command Meanwhile, the interior of the vehicle 1 may include a center console 110 disposed between the seats 51 and 52 and a tray 112 connected to the center console 110. The center console 110 may include a gear lever 111 and various input buttons 113 of a jog shuttle type or a key type, and the like.

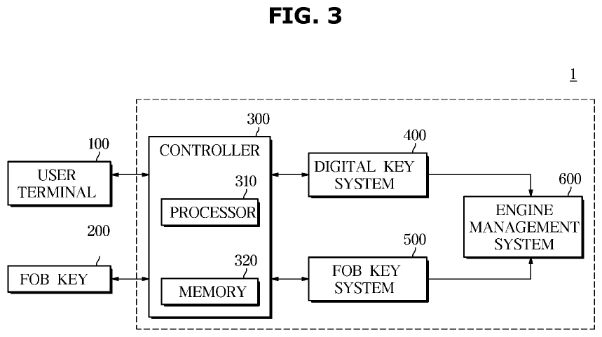

FIG. 3 is a control block diagram of a vehicle according to one exemplary embodiment of the disclosed invention. The vehicle 1 may include a digital key system 400 and a fob key system 500 configured to perform wireless communication from the user terminal 100 and the fob key 200. Therefore, the vehicle 1 may be configured to perform user authentication based on the authentication code received from the user terminal 100 or the fob key 200. In particular, if the authentication code is valid, the door of the vehicle 1 may be locked or released, and the vehicle 1 may allow the power and the driving device of the vehicle 1 to be activated, including starting.

The user terminal 100 refers to an electronic device capable of wireless communication and installing an application for operating the vehicle 1. For example, the user terminal 100 may include any type of handheld based wireless communication device such as a Personal communication system (PCS), a Global System for Mobile communications (GSM), a Personal Digital Cellular (PDC), a Personal Handyphone System (PHS), a Personal digital assistant (PDA), an International Mobile Telecommunication (IMT)-2000, a Code Division Multiple Access (CDMA)-2000, a W-Code Division Multiple Access (W-CDMA), a WiBro (Wireless Broadband Internet) terminal and a smart Phone. The user terminal may be configured to perform Near Field Communication (NFC) communication or Bluetooth Low Energy (BLE) communication with the digital key system 400 provided in the vehicle 1.

According to an exemplary embodiment, the user terminal 100 may include a alcohol drinking measurement module (not shown) capable of determining an alcohol drinking status of the user, and configured to output information regarding an alcohol drinking status obtained in the alcohol drinking measurement module. A breathalyzer application capable of analyzing alcohol decomposition time based on the user's body information may be installed in the user terminal 100. Therefore, the user terminal 100 may be configured to perform alcohol drinking authentication. In particular, the alcohol drinking measurement module may be a current sensor configured to measure the magnitude of the current generated when the alcohol is oxidized. The alcohol drinking measurement module is provided at the time of manufacture integrally provided in the user terminal 100, or may include a variety of sensors that may be connected to the user terminal 100 via a connector. In addition, the breathalyzer application installed in the user terminal 100 may be a program that may be configured to determine the alcohol drinking status through image processing and machine learning of user's face, pupil, etc.

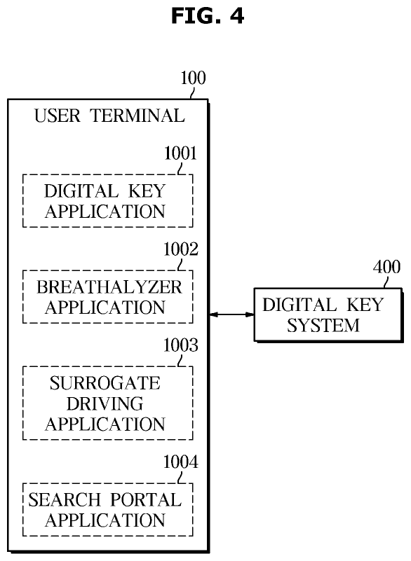

However, the alcohol drinking status of the user may be determined by an alcohol drinking measurement module provided in a space close to the user such as an AVN device and a steering wheel of the vehicle 1 in addition to the determination through the alcohol drinking measurement module or breathalyzer application provided in the user terminal 100 described above. Meanwhile, in addition to the breathalyzer application described above, various applications may be installed in the user terminal 100. In this regard, various applications installed in the user terminal 100 will be described with reference to FIG. 4. FIG. 4 is a view illustrating an application installed in the user terminal according to an exemplary embodiment of the present invention. As described above, the user terminal 100 may have a breathalyzer application 1002 installed therein.

A digital key application 1001 may be installed in the user terminal 100 according to an exemplary embodiment. The digital key application 1001 corresponds to an interface for operating the digital key system 400 mounted on the vehicle 1. The user may perform functions such as user authentication, locking or unlocking the door of the vehicle 1, remote start control, remote driving control, etc. through the digital key application 1001 installed in the user terminal 100.

Meanwhile, at least one of a digital key application 1001, a breathalyzer application 1002, a surrogate driving application 1003, and a search portal application 1004 may be installed in the user terminal 100 according to an exemplary embodiment. An interworking operation may be performed between the digital key application 1001 and the remaining applications 1002, 1003, and 1004. The breathalyzer application 1002 may be linked to the surrogate driving application 1003 and/or the search portal application 1004. For example, the user may interact with the surrogate driving application 1003 installed in the user terminal 100 through the setting of the digital key application 1001 to call the surrogate driver.

The surrogate driving application 1003 indicates an application that may search for, recommend, or call a surrogate driver according to a surrogate driving request of a user. Accordingly, the user may request a surrogate driving from the surrogate driver close to or in the vicinity of the vehicle 1 or the user terminal 100 through the surrogate driving application 1003. The surrogate driving application 1003 may be linked with at least one of the remaining applications 1002, 1003, and 1004 to allow the driver to call the surrogate driver making a direct surrogate driving request to the surrogate driving application 1003. The search portal application 1004 refers to an application installed in the user terminal 100 and capable of searching various information. For example, it may include various known search engines.

The fob key 200 refers to a signal generator configured to generate frequency signals of various bands to perform bidirectional communication with the fob key system 500 provided in the vehicle 1. For example, the fob key 200 may cause the fob key system 500 to operate based on the signal by providing signals to the fob key system 500 for user authentication of the vehicle 1, remote control of the locking device (e.g. door), remote start, etc. The fob key system 500 may be provided in the vehicle 1 to be configured to wirelessly communicate with the fob key 200 to open and close the door of the vehicle 1, and may unlock the door after user authentication. In addition, the fob key system 500 may be connected to the engine management system 600 to control whether the engine of the vehicle 1 is operated.

The controller 300 may be an electronic controller unit (ECU) configured to execute overall operations of the vehicle 1, and operate various modules, devices, and the like embedded in the vehicle 1. The controller 300 may be operated by at least one processor 310 embedded in the vehicle 1, and may be configured to generate a control signal for operating various modules, devices, etc. built in the vehicle 1 to execute the function of each component.

In addition, the controller 300 may use a controller area network (CAN) network of the vehicle 1. The CAN network refers a network system used for data transmission and control between ECUs of the vehicle 1. Specifically, the CAN network transmits data through two stranded data wires that are twisted or shielded by a sheath. The CAN works according to the multi-master principle, in which multiple ECUs perform the master function in a master/slave system. In addition, the controller 300 may be configured to communicate via an in-vehicle wired network such as a local interconnect network (LIN), a media oriented system transport (MOST), or a wireless network such as Bluetooth.

The memory 320 may be configured to store data or software/firmware or the like necessary for the controller 300 to perform control. In particular, the memory 320 may be configured to store a driving schedule of the vehicle 1 or information regarding an NFC-accessible user terminal 100. In addition, authentication codes for the user terminal 100 and the fob key 200 may be stored. The memory 320 may be implemented using at least one of a non-volatile memory element, e.g., a cache, a Read Only Memory (ROM), a Programmable ROM (PROM), an Erasable Programmable ROM (EPROM), an Electrically Erasable Programmable ROM (EEPROM) and a flash memory, a volatile memory element, e.g., a Random Access Memory (RAM), or a storage medium, e.g., a Hard Disk Drive (HDD) and a CD-ROM. The implementation of the memory is not limited thereto. The memory 320 may be implemented as a separate chip from the controller 300, or may be implemented as a single chip with the processor 310.

The digital key system 400 may be configured to receive the authentication code stored in the user terminal 100 and determine whether the authentication code is valid. When the authentication code stored in the user terminal 100 is found to be valid, the digital key system 400 causes the user terminal 100 to perform an alcohol drinking authentication process. In response to determining that the stored authentication code is invalid, the digital key system 400 may be configured to output a notification to the user that a valid authentication code has not been recognized.

The engine management system 600 may be configured to adjust driving of an engine (motor in the case of an electric vehicle) and fuel injection of the vehicle 1. In particular, the engine management system 600 may adjust the engine driving of the vehicle 1 to be allowed or blocked according to a control signal by the controller 300. In addition, the engine management system 600 may restrict the driving of the driver by blocking fuel injection.

The vehicle 1 according to an exemplary embodiment may include a digital key system 400 configured to receive a result of alcohol drinking authentication from a user terminal 100 having at least one application installed therein. The vehicle 1 according to an exemplary embodiment may further include an engine management system 600 configured to generate a control signal for activating or deactivating engine driving of the vehicle 1 based on the result of the alcohol drinking authentication obtained by the digital key system 400 through the user terminal 100. The controller 300 connected to the digital key system 400 and the engine management system 600 may be configured to receive the results for the alcohol drinking status of the first driver based on the user terminal 100 performing the alcohol drinking authentication, initiate a call to the second driver through the linked surrogate driving application 1003 when the first driver is determined to be in an alcohol drinking status, receive the result of alcohol drinking status of the second driver and activate or deactivate the engine based on the alcohol drinking status of the second driver.

At this time, the first driver indicates a user who possesses the user terminal 100 and holds a valid authentication code for the vehicle 1. In addition, the second driver may be a substitute driver for the first driver. For example, the second driver may correspond to a surrogate driver registered in the surrogate driving application 1003. On the other hand, in the above, each function and operation of the configuration for performing control of the vehicle 1 has been described through a control block diagram. Hereinafter, a control method performed based on the above-described configuration will be described in detail.

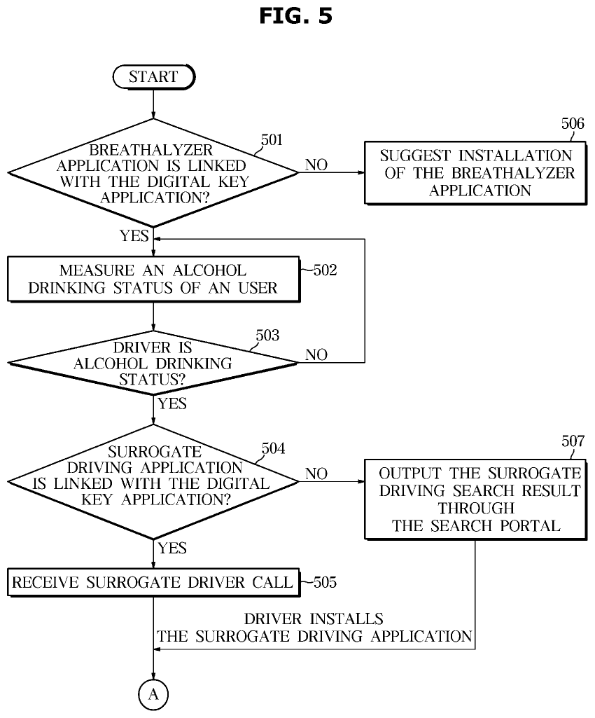

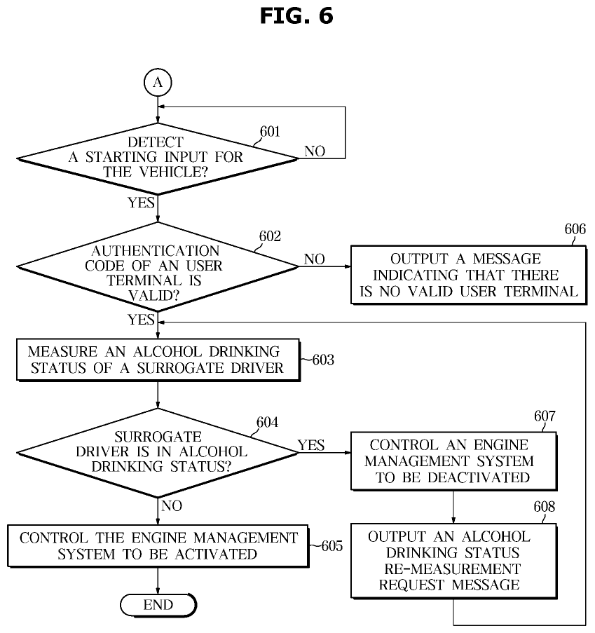

FIGS. 5 and 6 are flowcharts of a control method of a vehicle according to an exemplary embodiment of the disclosed invention. However, this is merely an exemplary embodiment for achieving the object of the present invention, of course, some steps may be added or deleted as necessary. The disclosed invention may be carried out independently or continuously with the control method according to FIGS. 5 and 6. First, a description will be given with reference to FIG. 5.

The controller 300 may be configured to determine whether the breathalyzer application 1002 is linked with the digital key application 1001 (501). At this time, in response to determining that the breathalyzer application 1002 is not linked, the controller 300 may be configured to output a message to the user terminal 100 or the AVN device 80 suggesting that the user install the breathalyzer application 1002. (506). When the breathalyzer application 1002 is installed but the interlocking setting is not established in the digital key application 100, the controller 300 may be configured to output a message indicating that the interlocking setting is not established. The user may refer to a driver possessing the user terminal 100, or may correspond to a passenger who is expected to driver the vehicle 1 in conjunction with the driver.

Further, the controller 300 may be configured to measure the alcohol drinking status of the user (502). In particular, the alcohol drinking status may be measured by the user terminal 100 in which the breathalyzer application 1002 is installed. According to an exemplary embodiment, the controller 300 may be configured to maintain the deactivation of the engine management system when the first driver is determined to be in an alcohol drinking status. Therefore, the disclosed invention may prevent the drunk driving of the user until the surrogate driver arrives or reaches the vehicle.

If the user who is the first driver is determined to be in an alcohol drinking status (503), the controller (300) may be configured to detect whether the surrogate driving application 1003 is linked with the digital key application (1001) (504), and when the digital key application 1001 is linked with the surrogate driving application 1003, the controller 300 may be configured to initiate a call to the surrogate driver. Thereafter, when the controller 300 detects that the second driver who is the surrogate driver arrives at the vehicle 1, the controller 300 may be configured to operate the engine management system 600 to be changed from deactivation to activation.

When the surrogate driving application 1003 is not linked with the digital key application 1001, the controller 300 may be configured to automatically output the surrogate driving search result through the search portal (507). Accordingly, the user may install the surrogate driving application 1003 in the user terminal 100 through the search portal. The controller 300 may be configured to receive an input for a surrogate driver call from a user (505) and initiate a call to a registered surrogate driver through an installed surrogate driving application 1003.

A description will now be given with reference to FIG. 6. The controller 300 may be configured to detect a starting input for the vehicle 1 (601). In particular, the starting input may be by a user who is a first driver or by a surrogate driver who is a second driver. When the authentication code of the user terminal 100 is valid (602), the controller 300 may be configured to operate the user terminal 100 to proceed with the procedure of measuring (603) the alcohol drinking status of the surrogate driver in preparation for the driving of the vehicle 1. At this time, when the authentication code is not valid, the controller 300 may be configured to output a message indicating that there is no valid user terminal 100 (606). The authentication code may be stored in the fob key 200 in addition to the user terminal 100.

As a result of performing step 603, when the controller 300 determines that the surrogate driver is in an alcohol drinking status (604), the controller 300 may be configured to block the surrogate driver who is in the alcohol drinking status from driving the vehicle 1 by operating the engine management system 600 to be deactivated (607). At this time, the controller 300 may be configured to output an alcohol drinking status re-measurement request message for the surrogate driver to correct the false measurement (608). On the contrary, when the controller 300 determines that the surrogate driver is not in the alcohol drinking status as a result of performing step 603 (604), the controller 300 may be configured to allow the surrogate driver to drive the vehicle 1 on behalf of the user by operating the engine management system 600 to be activated (605).

The vehicle according to an aspect of the disclosed invention may prevent drunk driving in advance from user operation of the vehicle, and may force a request for surrogate driving in the alcohol drinking status. This primary effect allows new business models to be built.

Meanwhile, the disclosed exemplary embodiments may be implemented in the form of a recording medium storing instructions that are executable by a computer. The instructions may be stored in the form of a program code, and when executed by a processor, the instructions may generate a program module to perform operations of the disclosed exemplary embodiments. The recording medium may be implemented as a non-transitory computer-readable recording medium. The non-transitory computer-readable recording medium may include all types of recording media storing commands that can be interpreted by a computer. For example, the non-transitory computer-readable recording medium may be ROM, RAM, a magnetic tape, a magnetic disc, flash memory, an optical data storage device, etc.

The exemplary embodiments of the disclosure have thus far been described with reference to the accompanying drawings. It will be obvious to those of ordinary skill in the art that the disclosure may be practiced in other forms than the exemplary embodiments as described above without changing the technical idea or essential features of the disclosure. The above exemplary embodiments are only by way of example, and should not be interpreted in a limited sense.

What is claimed is:

1. A vehicle, comprising:
a digital key system configured to receive a result of alcohol drinking authentication from a user terminal in which at least one application is installed;
an engine management system configured to generate a control signal for activating or deactivating engine driving of the vehicle based on the result of the alcohol drinking authentication; and
a controller including at least one processor;
wherein the controller is configured to receive a result for an alcohol drinking status of a first driver based on the alcohol drinking authentication performed by the user terminal, initiate a call to a second driver when the first driver is determined to be in the alcohol drinking status, receive a result of the alcohol drinking status of the second driver and operate the engine management system according to the alcohol drinking status of the second driver.

2. The vehicle according to claim 1, wherein the at least one application includes a digital key application, a breathalyzer application, and a surrogate driving application and wherein the controller is configured to initiate the call to the second driver when the digital key application is linked with the breathalyzer application and the surrogate driving application.

3. The vehicle according to claim 1, wherein the at least one application includes a digital key application and wherein the controller is configured to suggest an installation of the breathalyzer application to the first driver when the digital key application is not linked with a breathalyzer application.

4. The vehicle according to claim 1, wherein the at least one application includes a digital key application and a breathalyzer application and wherein the controller is configured to output the surrogate driving site result through a search portal when the digital key application is not linked with the surrogate driving application.

5. The vehicle according to claim 1, wherein the controller is configured to receive a result of the alcohol drinking status of the second driver and deactivate the engine management system when the second driver is determined to be in the alcohol drinking status.

6. The vehicle according to claim 1, wherein the controller is configured to receive a result of the alcohol drinking status of the second driver and activate the engine management system when the second driver is determined not to be in the alcohol drinking status.

7. The vehicle according to claim 1, wherein the at least one application includes at least one of the group consisting of: a digital key application, a breathalyzer application, a surrogate driving application, and a search portal application.

8. The vehicle according to claim 1, wherein the controller is configured to maintain deactivation of the engine management system when the first driver is determined to be in the alcohol drinking status.

9. The vehicle according to claim 8, wherein the controller is configured to operate the engine management system to change from deactivation to activation in response to detecting that the second driver arrives at the vehicle.

10. A control method of a vehicle, comprising:
receiving, by a controller, a result of alcohol drinking authentication from a user terminal in which at least one application is installed;
generating, by the controller, a control signal for activating or deactivating engine driving of the vehicle based on the result of the alcohol drinking authentication;
receiving, by the controller, a result for an alcohol drinking status of a first driver based on the alcohol drinking authentication performed by the user terminal;
initiating, by the controller, a call to a second driver when the first driver is determined to be in the alcohol drinking status;
receiving, by the controller, a result for the alcohol drinking status of the second driver; and
operating, by the controller, the engine management system according to the alcohol drinking status of the second driver.

11. The control method according to claim 10, wherein the at least one application includes a digital key application, a breathalyzer application, and a surrogate driving application and wherein the initiating of a call to the second driver includes:
initiating, by the controller, the call to the second driver when the digital key application is linked with the breathalyzer application and the surrogate driving application.

12. The control method according to claim 10, wherein the at least one application includes a digital key application and wherein the initiating of a call to the second driver includes:
suggesting, by the controller, an installation of a breathalyzer application to the first driver when the digital key application is not linked with the breathalyzer application.

13. The control method according to claim 10, wherein the at least one application includes a digital key application and a breathalyzer application and wherein the initiating of the call to the second driver includes:
outputting, by the controller, the surrogate driving site result through a search portal when the digital key application is not linked with the surrogate driving application.

14. The control method according to claim 10, wherein the operating of the engine management system includes:
receiving, by the controller, a result of the alcohol drinking status of the second driver and deactivating, by the controller, the engine management system when the second driver is determined to be in the alcohol drinking status.

15. The control method according to claim 10, wherein the operating of the engine management system includes:
receiving, by the controller, a result of the alcohol drinking status of the second driver and activating, by the controller, the engine management system when the second driver is determined not to be in the alcohol drinking status.

16. The control method according to claim 10, wherein the at least one application includes at least one of the group consisting of: a digital key application, a breathalyzer application, a surrogate driving application, and a search portal application

17. The control method according to claim 10, wherein the operating of the engine management system includes:
maintaining, by the controller, deactivation of the engine management system when the first driver is determined to be in the alcohol drinking status.

18. The control method according to claim 17, wherein the operating of the engine management system includes:
operating, by the controller, the engine management system to change from deactivation to activation when detecting that the second driver arrives at the vehicle.

19. A non-transitory computer readable recording medium comprising program causing the computer to execute steps of:
receiving a result of alcohol drinking authentication from a user terminal in which at least one application is installed;
generating a control signal for activating or deactivating engine driving of the vehicle based on the result of the alcohol drinking authentication;
receiving a result for an alcohol drinking status of a first driver based on the alcohol drinking authentication performed by the user terminal;
initiating a call to a second driver when the first driver is determined to be in the alcohol drinking status;
receiving a result for alcohol drinking status of the second driver; and
operating the engine management system according to the alcohol drinking status of the second driver.

20. The non-transitory computer readable recording medium according to claim 19, wherein the at least one application includes a digital key application, a breathalyzer application, a surrogate driving application and a search portal application.
